(19) United States
(12) Patent Application Publication
(10) Pub. No.: US 2009/0105118 A1
(43) Pub. Date: Apr. 23, 2009

(54) Preparation and applications of novel complexes made by gamma-polyglutamic acid and cisplatin

(76) Inventors: Haifeng Ye, Zurich (CH); Zirong Wu, Shanghai (CN); Jing Huang, Shanghai (CN); Li Jin, Shanghai (CN); Rongzhang Hu, Shanghai (CN); Chengliang He, Shanghai (CN); Jian Liu, Shanghai (CN)

Correspondence Address: BUTZEL LONG;IP DOCKETING DEPT, 350 SOUTH MAIN STREET, SUITE 300, ANN ARBOR, MI, 48104, US

(21) Appl. No.: 11/975,507

(22) Filed: Oct. 19, 2007

(51) Int. Cl. A61K 38/02; C07K 2/00; A61P 35/00

(52) U.S. Cl. 514/2; 530/300

(57) ABSTRACT

A series of complexes made by γ-polyglutamic acid (γ-PGA) and cisplatin, their preparation and applications in biomedical field, specifically in cancer treatment. The complexes may be made by binding free cisplatin on small molecule γ-PGA through the reaction between carboxylic group of γ-PGA and Cl of cisplatin. The complexes show effective anticancer effect and are easy and cost effective to prepare.

### TECHNICAL FIELD

The present invention relates to a series of complexes made from γ-polyglutamic acid (γ-PGA) and cisplatin, their preparation and applications in biomedical fields, particularly in cancer treatment.

### BACKGROUND ART

γ-PGA is a polymer produced by microbial; it can be made by the condensation between the α-amino group and γ-carboxylic group of glutamic acid. γ-PGA was first discovered by Ivanovics et al. (Ivanovics et al., Immunit atsforch. 1937, 90, 304-318) from Bacillus Licheniformis. Subsequently, Bovarnick et al. (Biol Chem. 1942, 145:415) discovered that γ-PGA can be produced by fermentation of some Bacillus Licheniformis. The molecular weight of γ-PGA produced by fermentation ranges between 10 kD and 2000 kD and may be made and separated based on molecular weight ranges for different uses.

γ-PGA consists of a great number of carboxylic groups and is known to form complexes with certain drugs. The complexes can be metabolized to become glutamic acid and release the drugs. Therefore, γ-PGA is an ideal vehicle for drug delivery. Because it does not accumulate and has no significant toxicity, γ-PGA has been used in slow release drugs and as a disease-targeting vehicle.

The main medical application of γ-PGA is in slow release drugs, as a disease-targeting vehicle, and as a vehicle for topical drugs. γ-PGA can also be used in non-permanent implantation devices. Besides its desirable physical and chemical properties, γ-PGA's biological compatibility is also desirable. A representative example is the anticancer drug by Cell Therapeutics (CTI), made by γ-PGA and paclitaxel [PG-TXL (CT22103)]. In this case, γ-PGA is used to increase the distribution of paclitaxel in the cancerous area by increasing the solubility of paclitaxel. Lung cancer cells were reduced by 75% for mice injected with 120 mg/kg of PG-TXL. However, when paclitaxel was used alone, only a 58% reduction was observed. In addition, drug resistance was also reduced when PG-TXL was used.

Currently, most γ-PGA polymers are made by synthetic methods which produce low yields and have high production costs which limit commercialization.

Cisplatin (cis-daimminedichloroplatinum(II), CDDP) is cytoxic. It forms a chelation with DNA and hinders the replication of DNA. It has obvious anticancer activity and has been used as an effective anticancer drug for over twenty years. It has been used in treating ovarian cancer, small cell lung cancer, non-small cell lung cancer, head and neck cancer, GI track cancer, seminoma, bladder cancer, mesothelioma etc. However, cisplatin has a very low solubility, low selectivity, and significant toxicity. Therefore, clinical use of cisplatin significantly limited.

### DISCLOSURE OF THE INVENTION

According to various features, characteristics and embodiments of the present invention which will become apparent as the description thereof proceeds,

### BRIEF DESCRIPTION OF DRAWINGS

The present invention will be described with reference to the attached drawings which are given as non-limiting examples only, in which:

FIG. 1 is a Nexus 670 IR spectrum of γ-PGA obtained by fermentation.

FIG. 2 is an AVANCE 500 MHz ¹³C NMR spectrum of γ-PGA obtained by fermentation.

FIG. 3 is an AVANCE 500 MHz ¹H NMR spectrum of a γ-PGA obtained by fermentation.

FIG. 4 is gel electrophoresis results of a γ-PGA obtained by fermentation. Sample #1 is a standard of γ-PGA purchased from Sigma, mw 97 kD. Sample #2 is another standard of the γ-PGA, mw 13 kD. Sample #3 is the small molecule γ-PGA produced by the present invention. Sample #4 is the large molecule γ-PGA produced by the present invention (un-degraded) as a reference.

FIG. 5 is a comparison of the cytotoxicity of γ-PGA-CDDP and CDDP on ovarian cancer cells OVCAR-3 after 24 h.

FIG. 6 is a comparison of the cytotoxicity of γ-PGA-CDDP and CDDP on liver cancer cells BEL-7402 after 48 h.

FIG. 7 is a comparison of the cytotoxicity of γ-PGA-CDDP and CDDP on breast cancer cells Bcap-37 after 24 h.

FIG. 8 is a comparison of the cytotoxicity of γ-PGA-CDDP and CDDP on breast cancer cells Bcap-37 after 48 h.

FIG. 9 is a comparison of the cytotoxicity of γ-PGA-CDDP and CDDP on breast cancer cells Bcap-37 after 72 h.

FIG. 10 is a comparison of the toxicity of γ-PGA-CDDP and CDDP based on the survival of China KM mice.

FIG. 11 is a comparison of the toxicity of γ-PGA-CDDP and CDDP based on the body weight of China KM mice.

FIG. 12 is a comparison of the toxicity of γ-PGA-CDDP and CDDP based on the survival of BALB/C female mice.

FIG. 13 is a comparison of the toxicity of γ-PGA-CDDP and CDDP based on the body weight of BALB/C female mice.

FIG. 14 shows the effect of γ-PGA-CDDP and CDDP on tumor volume after one injection.

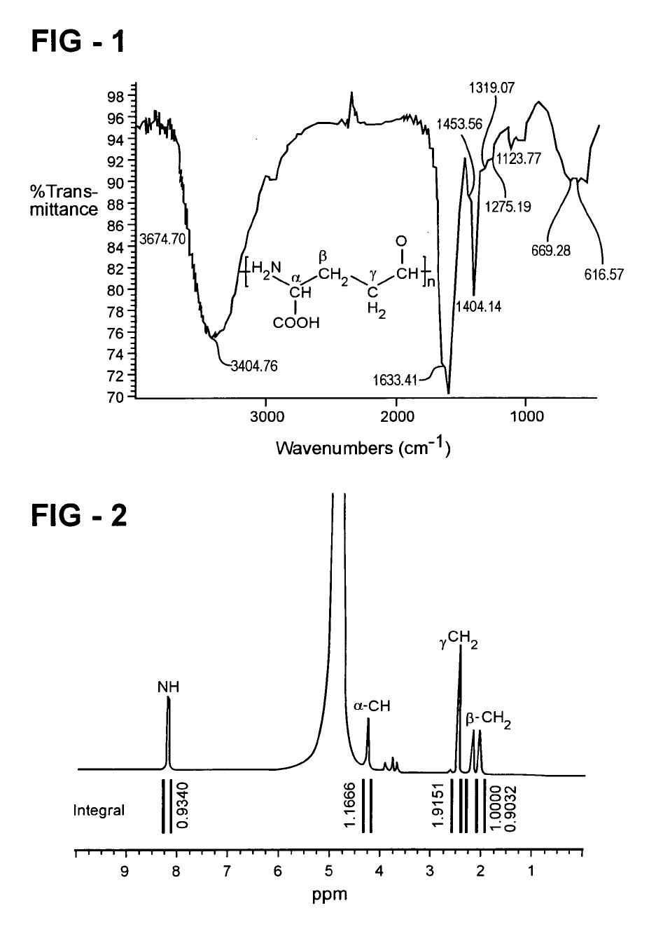

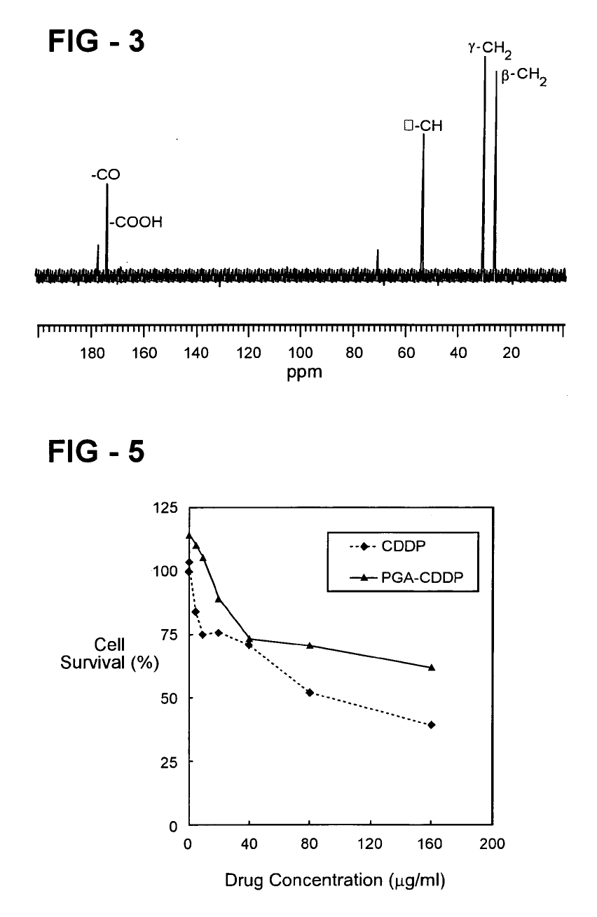

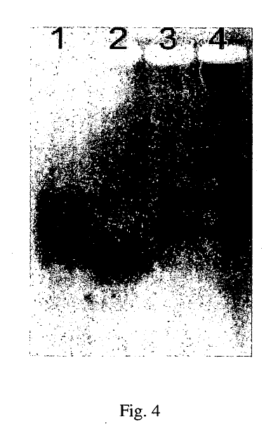

### BEST MODE FOR CARRYING OUT THE INVENTION

The present invention is directed to a series of complexes made from γ-polyglutamic acid (γ-PGA) and cisplatin, their preparation and applications in biomedical fields, particularly in cancer treatment. In addition, the present invention provides a drug delivery vehicle for cisplatin which utilizes γ-PGA, to increase the water solubility of cisplatin and reduce its toxicity (or side effects). By forming a complex with γ-PGA, the complex has certain disease targeting properties based on the characteristic EPR effect of cancer tissues. The present invention also provides a simple and economic method to prepare the complex made by γ-PGA and cisplatin as well as methods for increasing the yields and/or lowering the cost of γ-PGA. The present invention is further directed to other uses of γ-PGA.

According to the present invention a complex is formed between γ-PGA and cisplatin. The binding of this complex is due to the reaction between the —COOH group of γ-PGA and Cl of cisplatin. A general formulation for the complex may be represented by the following:

(cisplatin)ₘ-(γ-PGA)

where m is an integer between 5 and 50 and γ-PGA has a molecular weight that ranges from 5 kD and 100 kD.

The complex may be represented by the following general structures:

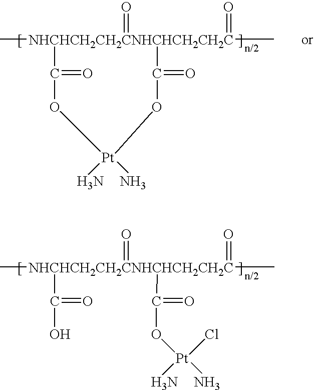

Wherein n is an integer between 40 and 800.

The present invention further is directed to a complex formed between γ-PGA and cisplatin in which the molecular weight of γ-PGA ranges from 20 kD to 80 kD. In further embodiments, the present invention is directed to a complex formed between γ-PGA and cisplatin in which the molecular weight of the γ-PGA in the complex ranges form 35 kD to 60 kD.

In the present invention, γ-PGA comprises both small molecule γ-PGA and large molecule γ-PGA, wherein large molecule γ-PGA has a molecular weight ranges between 1000 kD and 2000 kD, having the following structure:

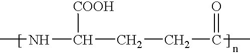

The small molecule γ-PGA is suitable for drug delivery with a molecular weight range between 5 kD and 100 kD. The small molecular γ-PGA may be obtained by degradation of large molecular γ-PGA or purchased directly form suppliers. The small molecular γ-PGA used in the present invention was purchased from Sigma-Aldrich.

The structure of cisplatin is shown below:

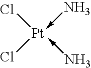

Preparation of the γ-PGA and cisplatin complex during the course of the present invention involved dissolving small molecule γ-PGA and cisplatin in double distilled water, mixing thoroughly, and separating the complex formed by γ-PGA and cisplatin.

The γ-PGA and cisplatin complex can also be formed by to dissolving cisplatin and silver nitrate in double distilled water, removing the precipitate, adding small molecule γ-PGA to the filtrate, mixing well, and separating the complex formed between the γ-PGA and cisplatin.

Another method of preparing the γ-PGA and cisplatin complex is to degrade large molecule γ-PGA to form small molecule γ-PGA (or purchase directly form a vendor, such as Sigma) and then react small molecule γ-PGA with cisplatin.

Large molecule γ-PGA may be made by certain biotechnologies, including through the fermentation of Bacillus Licheniformis ATCC 9945a. The large molecule γ-PGA can then be degraded to produce small molecule γ-PGA which can be used to form the complex with cisplatin. The cell culture used to produce large molecule γ-PGA includes maltose (50 g/l), yeast extract (10 g/l), glutamic acid (30 g/l), NaCl (10 g/l), KH₂PO₄ (5 g/l), and MgSO₄.7H₂O (0.5 g/l). After separation and purification, γ-PGA is obtained. The separation and purification methods include organic solvent precipitation. Centrifuging is used to remove bacterial and ethanol, propanol or acetone is added to the supernatant (2-5 times the volume of the supernatant) to obtain large molecule γ-PGA as a precipitate. Distilled water is used to dissolve the precipitate. Un-dissolved particulates are filtered off. Next dialysis is used to remove small molecules. And the remaining product is lyophilized to obtain pure γ-PGA as white crystalline powder. The small molecule γ-PGA can then be obtained by degradation in acid.

The complex in the present invention is made from small molecule γ-PGA, which can be made as follows. 2% of large molecule γ-PGA (prepared above) is dissolved in water. The same volume of 0.05 N HCl is added and the resulting solution is mixed mix well. (Alternatively HCl can be added directly until the pH is 2-3). The mixture is subjected to a high temperature and high pressure (121° C. and 0.1 MPa) for 10-20 min (or water bath for 30-100 min) and then cooled down in ice bath while adjusting the pH to 7-8. Subsequently dialysis and lyophilize is used to obtain small molecule γ-PGA as crystalline while powder.

The small molecule γ-PGA used in the present invention has the following characteristics:
The molecular weight range of the small molecule γ-PGA is 5 kD˜100 kD as determined by SDS-PAGE and gel electrophoresis.
The small molecule γ-PGA has more active groups and is easier to form complexes with drug molecules.

The present invention provides a method of treating ovarian cancer, breast cancer, liver cancer, lung cancer, stomach cancer, cervix cancer, prostate cancer, and bladder cancer by use of the complex of the small molecule γ-PGA and cisplatin.

The loading of cisplatin on the small molecule γ-PGA may be expressed as percentage (w/w) of cisplatin relative to the total weight of the complex.

Through the complexation with the small molecule γ-PGA, the efficacy of cisplatin is not reduced. This is shown in the examples below. In addition, cisplaitn becomes more soluble once bound onto the small molecule γ-PGA. Also, the distribution of ciaplatin in cancerous areas is significantly increased using the complex of the small molecule γ-PGA and cisplatin. Therefore, the total effect associated with using the complex of the small molecule γ-PGA and cisplatin is much better as compared to using free cisplatin alone.

The complexes disclosed in the present invention may be made by binding free cisplatin on the small molecule γ-PGA through the reaction between carboxylic group of γ-PGA and Cl of cisplatin. To be exact, the hydrogen on the carboxylic acid group is removed and a coordination bond is formed between oxygen and Pt⁺².

During the course of the present invention it was discovered that the un-reacted cisplatin must be removed form the complex to ensure the high efficacy and low toxicity of the complex. The un-reacted cisplatin may be removed by dialysis. The aliquot is then lyophilized to obtain white crystalline powder.

Spectrophotometer may be used to measure the amount of cisplatin in the complex. Under boiling water bath, cisplatin can form a blue solution with o-phenylene diamine (OPDA) and may be measured at 703 nm. A standard curve may be made and used to measure the amount of cisplatin loaded on the small molecule γ-PGA.

The complex formed between small molecule γ-PGA and cisplatin produced according to the present invention shows significant improvement of the anticancer activity and solubility as well as reduction of the toxicity. Further it was unexpectedly discovered during the course of the present invention that the complex formed between small molecule γ-PGA and cisplatin shows certain disease targeting effect based on its effect on EPR, which is characteristic of cancerous tissue.

The complex disclosed in the present invention is easy to prepare and economic in cost

The following are some practical examples:

### EXAMPLE 1

### (1) Method of Preparing γ-PGA-CDDP Complex

11.4 mg AgNO₃ and 20 mg cisplatin were dissolved in 10 ml double distilled water. The resulting solution was allowed to sit for 24 h. then centrifuged to remove the precipitate and filtered through a 0.22 μm membrane to remove fine particulates. 75 mg of small molecule γ-PGA was added to the resulting solution and the pH was adjusted to 7-8. The resulting mixture was mixed at 37° C. for 48 h under no light and then dialyzed for 24 hr to remove un-reacted cisplatin. Lyophilize was used to obtain the γ-PGA-CDDP complex as a white crystalline powder. Based on this procedure, the mole ratio of cisplatin and γ-PGA is 25:1, or m=25. This method of preparing γ-PGA-CDDP complex was used in all of the following examples.

11.4 mg AgNO₃ and 30 mg cisplatin were dissolved in 10 ml double distilled water and was allowed to sit for 24 h. then centrifuged to remove the precipitate and filtered through 0.22 μm membrane to remove fine particulates. Add 75 mg of small molecule γ-PGA added to the resulting solution and the pH was adjusted 7-8. The resulting mixture was mixed at 37° C. for 72 h under no light and then dialyzed for 16 hr to remove un-reacted cisplatin. Lyophilize was used to obtain the γ-PGA-CDDP complex as a white crystalline powder. Based on this procedure, the ratio of cisplatin and γ-PGA is 50:1, or m=50. This method of preparing γ-PGA-CDDP complex produces higher loading of cisplatin, but may be more costly.

10 mg cisplatin was dissolve in 10 ml double distilled water. 75 mg of small molecule γ-PGA was added to the solution which was allowed to sit for 2 h at room temp before being dialyzed for 24 h to remove un-reacted cisplatin. Lyophilize was used to obtain the γ-PGA-CDDP complex as a white crystalline powder. Based on this procedure, the ratio of cisplatin and γ-PGA is 5:1, or m=5. This method of preparing γ-PGA-CDDP complex produces lower loading of cisplatin.

### (2) Analysis of Cisplatin in γ-PGA-CDDP Complex

The γ-PGA-CDDP complex (containing 3-12 μg cisplatin) was dissolved in 0.6 ml double distilled water. The resulting solution was mixed with 0.6 ml of DMSO (dimethyl sulphoxide) solution of OPDA (o-phenylene diamine) (1.2 mg/ml). The resulting mixture was left in a boiling water bath for 10 min and immediately measured for absorption at 703 nm. A standard curve was used to quantify the amount of cisplatin in γ-PGA-CDDP complex.

### EXAMPLE 2

### (1) Use Fermentation to Obtain Large Molecule γ-PGA

Activate Bacillus Licheniformis ATCC 9945a, was inoculated to a slant culture and incubated at 37° C. for 11 h. The culture includes peptone (10 g/l), NaCl (5 g/l), yeast extract (5 g/l), and agar (20 g/l).

Culturing: Inoculate the activated bacterial in the cell culture, 37° C., 210 rpm.

Culturing medium: peptone (10 g/l), NaCl (5 g/l), yeast extract (5 g/l).

Fermentation medium: peptone (50 g/l), yeast extract (10 g/l), glutamic acid (30 g/l), NaCl (10 g/l), KH₂PO₄ (5 g/l), and MgSO₄.7H₂O (0.5 g/l). The fermentation medium was used to fill in a 200 ml/1000 ml shaking bottle and sterilized at 115° C. for 20 min. After sterilization and cool down, the medium was inoculated with 5% of Bacillus Licheniformis ATCC 9945a and cultured at 37° C. and rotated at 220 rpm. Fermentation was conducted for 48 h.

After fermentation, distilled water was added to the fermentation aliquot (4 times dilution), the pH was adjusted to about 3 and the aliquot was centrifuged at 16000 rpm for 20 min to remove the bacterial. The pH of the supernatant was adjusted to about 8, and 3 times the volume of ethanol was added and mixed to obtain a precipitate. The precipitate was dissolved in water and un-dissolved particulates were filter off before subjecting the liquid to dialyze, and finally lyophilize to obtain a crystalline white powder of γ-PGA. The yield can reach 30 g/l.

### (2) Preparation of Small Molecule γ-PGA

The large molecule γ-PGA from step (1) in Example 2 was used to make a 2% solution. 5 ml of the large molecule γ-PGA was used to mix with same volume of 0.05 N HCl. The aliquot was treated in a boiling water bath for 20 min and cooled down in an ice bath and the pH was adjusted to 7-8 to quench the reaction. Gel electrophoresis was used to determine that the molecular weight was 20 kD-100 kD. Based on an Ubbelohde viscometer, the molecular weight was 35 kD-60 kD. Preparation of γ-PGA-CDDP complex was the same as in example 1.

### EXAMPLE 3

### Large-Scale Preparation of Small Molecule γ-PGA

A 2% aqueous solution of the large molecule γ-PGA was prepared and the pH was adjusted to 2˜3. The aliquot was subjected to high temperature and high pressure (121° C., 0.1 MPa) for 15, 20, and 30 min and immediately placed in ice bath. The pH was adjusted to 7˜8. The aliquot was dialyzed and lyophilized to obtain small molecule γ-PGA as a white crystalline powder. An Ubbelohde viscometer to measure the molecular weight of the small molecule γ-PGA which was found to be 80-100 kD, 35-60 kD, or 5-20 kD. Preparation of the small molecule γ-PGA-CDDP complex was the same as in Example 1.

### EXAMPLE 4

### Cytotoxicity Study of γ-PGA-CDDP Complex on OVCAR-3 Human Ovarian Cancer Cells in vitro

γ-PGA-CDDP complex and cisplatin were individually dissolved in RPMI 1640 medium (contains 20% of bovine serum) and filtered to sterilize. An appropriate medium was added to make a series of samples with a concentration gradient containing 0˜160 μg/ml of γ-PGA-CDDP complex or cisplatin (4-plicate), and 100 μl of the samples were placed in a 96 cell plate, each containing 1×10⁴ OVCAR-3 cells. Similar same samples were prepared without drug substance as the control group. The samples were placed in a CO₂ incubator for 24 h. and 20 μl of 5 mg/ml MTT solution was added to each cell after which the samples were left in the incubator again for additional 5 hr. The supernatant was carefully removed and 200 μl of dimethyl sulphoxide (DMSO) was added to each sample. The samples were then shaken for 10 min and the absorption at 570 nm was measured.

Using the control as the reference the average of 4 absorption readings at 570 nm was taken (as the cell viability) and plotted versus the concentration of the drug substance. The results indicate that at the same concentration the cytotoxicity of γ-PGA-CDDP complex is lower than that of CDDP. This indicates that γ-PGA-CDDP complex maintains its anticancer activity but functions as a slow release vehicle. See FIG. 5 which is a comparison of the cytotoxicity of γ-PGA-CDDP and CDDP on ovarian cancer cells OVCAR-3 after 24 h.

### EXAMPLE 5

### Cytotoxicity Study of γ-PGA-CDDP Complex on BEL-7402 Human Liver Cancer Cells in vitro

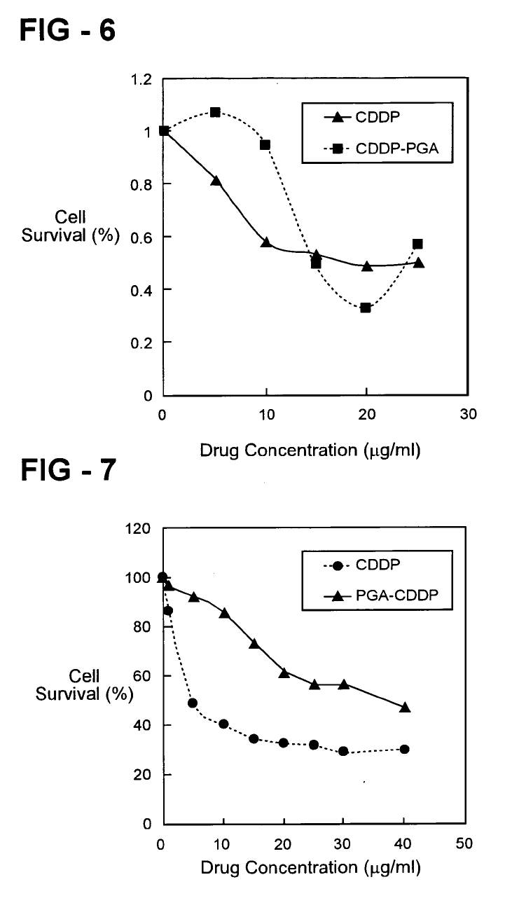

γ-PGA-CDDP complex and cisplatin individually dissolved in DMEM medium containing 10% bovine serum. The cisplatin concentration range was 0-25 μg/ml. All other procedures were the same as in Example 4 above. The results show that at 20 μg/ml the killing effect on BEL-7402 of γ-PGA-CDDP is higher than that of cisplatin in vitro. This indicates that the anticancer effect is not lost by the complexation. See FIG. 6 which is a comparison of the cytotoxicity of γ-PGA-CDDP and CDDP on liver cancer cells BEL-7402 after 48 h.

### EXAMPLE 6

### Cytotoxicity Study of γ-PGA-CDDP Complex on Bcap-37 Human Breast Cancer Cells in vitro

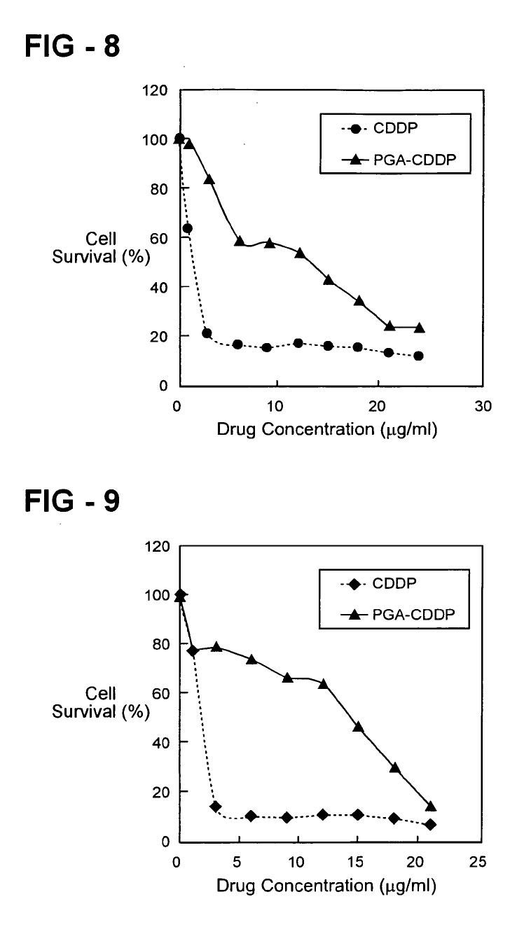

The same procedure as in example 4 was used and incubated with breast cancer cells Bcap-37 for 24, 48, and 72 h. to study the cancer cell killing effect in vitro. The results show that γ-PGA-CDDP complex has lower cytotoxicity against Bcap 37 in vitro at lower concentration. However, for the 48 and 72 samples, the results indicate that γ-PGA-CDDP complex maintains almost the same cytotoxicity against Bcap-37 as compared to cisplaitn. See FIG. 7, FIG. 8 and FIG. 9 which are comparisons of the cytotoxicity of γ-PGA-CDDP and CDDP on breast cancer cells Bcap-37 after 24 h., 28. and 27 h.

### EXAMPLE 7

### Cytotoxicity Study of γ-PGA-CDDP Complex in vivo

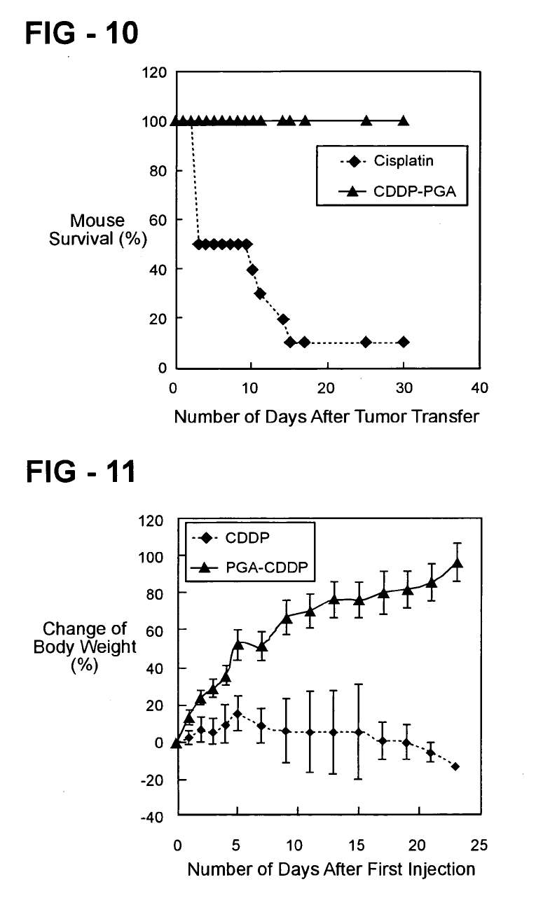

20 healthy Kun Ming mice were randomly divided (10 each for male and female) into two groups. Cisplatin and γ-PGA-CDDP was individually injected in each group in a tail vein. The injections were done at 0, 5, and 10 days at 5 mg/kg. The survival and body weight of the mice were recorded. The results are shown in FIG. 10 and FIG. 11 which are comparisons of the toxicity of γ-PGA-CDDP and CDDP based on the survival of China KM mice and based on the body weight of China KM mice.

As shown in FIG. 10, cisplatin has significant toxicity and 90% died after 15 days. On the other hand, γ-PGA-CDDP did not show significant toxicity and 100% survived after 30 days.

FIG. 11 shows the in vivo toxicity indicated by weight changes. The body weight was reduced by cisplatin indicating that cisplatin has significant toxicity in Kun Ming mice. On the other hand, the group treated by γ-PGA-CDDP actually showed increase of body weight indicating that its in vivo toxicity is not significant.

### EXAMPLE 8

### Effect of γ-PGA-CDDP Complex on a Xenographed Animal Model

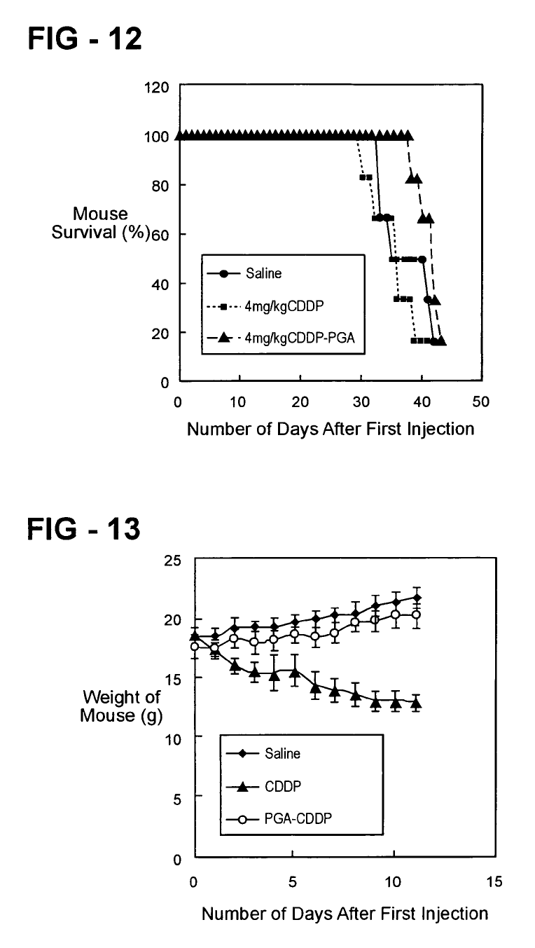

Human breast cancer cells Bcap-37 in DMEM with 10% bovine serum albumin were cultured at 37° C. and 5% CO₂. 5×106 Bcap cells were injected in each of BALB/C female SKID mouse (18 mice, 4 week old, 20 g each). After 7 days the mice were randomly divided into 3 groups, 6 mice per group. The first group was treated with saline, the second group was treated with cisplatin, and the third group was treated with γ-PGA-CDDP. γ-PGA-CDDP complex (at equivalent of 4 mg/kg cisplatin) was injected in a tail vein at 0 and 5 days. The survival and body weight was recorded. The results are shown in FIG. 12 and FIG. 13 which are comparisons of the toxicity of γ-PGA-CDDP and CDDP based on the survival and body weight of BALB/C female mice.

As shown in FIG. 12, γ-PGA-CDDP does increase the surviving time, but not cisplatin.

FIG. 13 shows that the group treated by γ-PGA-CDDP shows a gradual increased in body weight indicating that the toxicity of γ-PGA-CDDP is not significant. On the other hand, the group treated by cisplatin shows obvious decrease of body weight indicating that cisplatin has significant toxicity.

### EXAMPLE 9

### Effect of γ-PGA-CDDP Complex on a Xenographed Animal Model

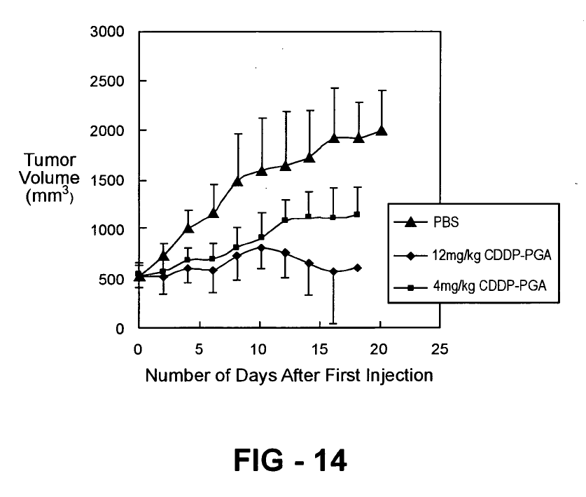

The same procedure in example 8 above was followed, but 24 mice were used and the mice were divided into three groups, of 8 mice in each group. The first group was used as a control (saline), the second group was treated with γ-PGA-CDDP (equivalent to 4 mg/kg cisplaitn), and the third group was treated with PGA-CDDP (equivalent to 12 mg/kg cisplatin). Injections were made in a tail vein on 0, 2, and 4 days. Tumor volume was recorded daily and the results are shown in FIG. 14.

As shown in FIG. 14, γ-PGA-CDDP shows clear inhibition on the tumor growth in vivo.

Although the present invention has been described with reference to particular means, materials and embodiments, from the foregoing description, one skilled in the art can easily ascertain the essential characteristics of the present invention and various changes and modifications can be made to adapt the various uses and characteristics without departing from the spirit and scope of the present invention as described above.

What is claimed is:

1. A γ-PGA-CDDP complex comprising γ-PGA and cisplatin (CDDP) having the general formula:
(Cisplatin)ₘ γ-PGA
wherein m is an integer between 5 and 50, γ-PGA is small molecule γ-Polyglutamic acid having a molecular weight between 5 kD and 100 kD.

2. A γ-PGA-CDDP complex according to claim 1, wherein the structural relationship between small molecule γ-PGA and cisplatin is characterized as: 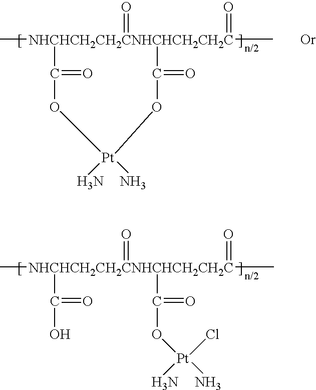
wherein n is an integer between 40 and 800.

3. A γ-PGA-CDDP complex according to claim 1, wherein the molecular weight of the small molecule γ-PGA is between 20 kD and 80 kD.

4. A γ-PGA-CDDP complex according to claim 1, wherein the molecular weight of the small molecule γ-PGA is between 35 kD and 60 kD.

5. A method of preparing the γ-PGA-CDDP complex having the general formula:
(Cisplatin)ₘ γ-PGA
wherein m is an integer between 5 and 50, γ-PGA is small molecule γ-Polyglutamic acid having a molecular weight between 5 kD and 100 kD,
said method comprising mixing the small molecule γ-PGA and cisplatin in double distilled water until reaction is complete and separating and purifying the resulting γ-PGA-CDDP complex.

6. A method of preparing γ-PGA-CDDP complex according to claim 1, wherein the method further comprises mixing cisplatin and silver nitrate in double distilled water, removing the resulting precipitate, adding the small molecule γ-PGA, mixing thoroughly till the reaction is complete, separating and purifying the resulting γ-PGA-CDDP complex.

7. A method of preparing γ-PGA-CDDP complex according to claim 5, wherein the small molecule γ-PGA is obtained by degradation of large molecule γ-PGA, wherein the large molecule γ-PGA is obtained by fermentation and the fermentation medium comprises maltose, yeast extract, sodium glutamate, NaCl, KH₂PO₄, and MgSO₄.7H₂O.

8. A method of preparing γ-PGA-CDDP complex according to claim 6, wherein the small molecule γ-PGA is obtained by degradation of large molecule γ-PGA, wherein the large molecule γ-PGA is obtained by fermentation and the fermentation medium comprises maltose, yeast extract, sodium glutamate, NaCl, KH₂PO₄ and MgSO₄.7H₂O.

9. A method of treating human cancer by administration of an effective amount of the complex in claim 1.

10. A method of treating human cancer according to claim 9, wherein the human cancer comprises ovarian cancer, breast cancer, lung cancer, stomach cancer, cervix cancer, prostate cancer, and bladder cancer.
